(19) United States
(12) Patent Application Publication
(10) Pub. No.: US 2024/0299380 A1
(43) Pub. Date: Sep. 12, 2024

(54) APPLICATION OF COMPOUND IN PREPARATION OF DRUG FOR TREATING MYELOFIBROSIS AND RELATED SYMPTOMS/SIGNS THEREOF, AND USE OF COMPOUND

(71) Applicant: Betta Pharmaceuticals Co., Ltd, Hangzhou (CN)

(72) Inventors: Jing Guo, Beijing (CN); Dan Yan, Beijing (CN); Yu Wang, Beijing (CN); Tong Xu, Beijing (CN); Kao Li, Beijing (CN); Wei Xu, Beijing (CN); Hong Lan, Beijing (CN); Lieming Ding, Hangzhou (CN); Jiabing Wang, Beijing (CN)

(21) Appl. No.: 18/572,432

(22) PCT Filed: Jun. 21, 2022
(86) PCT No.: PCT/CN2022/100134

§ 371 Date: Dec. 20, 2023

(30) Foreign Application Priority Data
Jun. 21, 2021 (WO) PCT/CN2021/101322

(51) Int. Cl. A61K 31/4985; A61P 35/00

(52) U.S. Cl. CPC A61K 31/4985; A61P 35/00

(57) ABSTRACT

The present invention provides an application of a compound in the preparation of drug for treating myelofibrosis and the related symptoms/signs thereof, and a use of the compound. The compound is selected form one or more of a compound having a structure as shown in formula I, and a pharmaceutically-acceptable salt, prodrug, solvate and hydrate thereof.

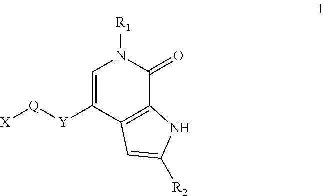

### FIELD OF THE INVENTION

The present invention relates to the field of medicament, specifically, relates to a use of a compound in the preparation of the medicament for treating myelofibrosis and the symptoms/signs associated with myelofibrosis, and the use thereof.

### BACKGROUND OF THE INVENTION

Myelofibrosis (MF) is a myeloproliferative neoplasm (MPNs) caused by the clonal proliferation of hematopoietic stem cells, including primary myelofibrosis, myelofibrosis secondary to polycythemia vera and essential thrombocythemi. Myelofibrosis (MF) is a disease characterized by the occurrence of reticular fibers and collagen fibers in the bone marrow, the clinical manifestations of MF include anemia, fatigue, early satiety, abdominal imbalance, poor mobility, difficulty concentrating, night sweats, itchy skin, diffuse bone pain, fever, weight loss, splenomegaly, hepatomegaly, which impacts quality of life seriously. The overall median survival time is 3.5 to 5.5 years, but the higher the risk level, the shorter the survival time, the median survival time of high-risk patients is about 2 years. The cause of myelofibrosis (MF) has not been well understood, most patients carry an activating mutation of the Janus kinase 2 gene (JAK2), CALR or MPL or other genes, which make the JAK-STAT pathway overreacted. About 85% of MF patients have at least one driver gene mutation among JAK, MPL and CALR (Klampfl T, et al. NEJM 2013; 369(25):2379-90; Nangalia J, et al. NEJM 2013; 369(25)2391-405). In patients with primary fibromyelopathy, mutational frequency of JAK2, CALR and MPL is ˜65%, ˜25% and ˜10% respectively. (Tefferi A. Am J Hematol. 2021 January; 96(1):145-162.).

The treatment of Myelofibrosis (MF) is to control disease symptoms and complications, enhancing quality of life in the short term, but in the long term the treatment is to extend survival, manage/reverse Myelofibrosis (MF) even cure it. Among the current treatments for MF, traditional drugs are symptomatic treatments based on clinical manifestations, and their efficacy is limited. Currently, there are few targeted therapies for myelofibrosis. Only two targeted drugs for the treatment of myelofibrosis have been developed, which are small molecule JAK1/JAK2 kinase inhibitor ruxolitinib phosphate from Novartis and highly specific JAK2 inhibitor fedratinib from Bristol-Myers Squibb's (BMS). At present, ruxolitinib is the only targeted drug approved in China for the treatment of myelofibrosis. But JAK inhibitors has shown some side effects in patients including thrombocytopenia, anemia, gastrointestinal disturbances, metabolic abnormalities, peripheral neuropathy, and hyperacute relapse of symptoms during treatment discontinuation. (Tefferi, A. JAK inhibitors for myeloproliferative neoplasms: clarifying facts from myths[J]. Blood, 2012, 119(12):2721-2730.).

For the above reasons, there is an urgent need to develop a drug that can effectively treat myelofibrosis and its related symptoms/signs.

### SUMMARY OF THE INVENTION

The present invention is to provide a use of a compound in the preparation of a medicament for treating myelofibrosis and the symptoms/signs associated with myelofibrosis, the use of the compound as a new medicament for the treatment of myelofibrosis and the symptoms/signs associated with myelofibrosis.

In order to achieve the above purpose, according to one aspect of the present invention, there is provided use of a compound in the preparation of a medicament for treating myelofibrosis and the symptoms/signs associated with myelofibrosis, wherein the compound is selected from one or more of the compounds of formula I and the pharmaceutically acceptable salts, prodrugs, solvates and hydrates thereof:

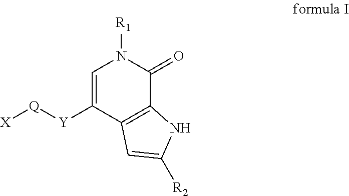

in the formula I,
R₁ and R₂ are each independently selected from H, C₁₋₆ alkyl, C₁₋₆ alkoxy, C₆₋₁₀ aryl or C₅₋₁₀ heteroaryl, wherein the C₁₋₆ alkyl, C₁₋₆ alkoxy, C₆₋₁₀ aryl or C₅₋₁₀ heteroaryl is optionally substituted with C₁₋₆ alkyl group, —NH₂ group. —OH group, C₆₋₁₀ aryl group or C₅₋₁₀ heteroaryl group; and the C₅₋₁₀ heteroaryl and the C₅₋₁₀ heteroaryl group have 1, 2, or 3 heteroatoms independently selected from nitrogen, oxygen or sulfur;
Q is absent or selected from C₁₋₆ alkylene, —SO₂— or —NH—, wherein the C₁₋₆ alkylene or —NH— is optionally substituted with halogen, C₁₋₆ alkyl or C₁₋₆ alkoxy;
X is selected from H, C₁₋₆ alkyl, C₆₋₁₀ aryl or C₅₋₁₀ heteroaryl, wherein the C₁₋₆ alkyl. C₆₋₁₀ aryl or C₅₋₁₀ heteroaryl is optionally substituted by halogen, halo C₁₋₆ alkyl, C₁₋₆ alkyl, C₁₋₆ alkoxy, C₁₋₆ alkylthio, C₁₋₆ alkoxycarbonyl or C₁₋₆ alkyl-SO₂—;

Y is

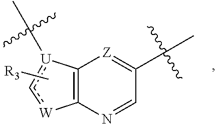

wherein,

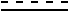

represents a single bond or a double bond, the U, W or Z is independently selected from C or N; R₃ is absent or selected from H, halogen, hydroxyl, amino, C₁₋₆ alkyl, C₁₋₆ alkoxy, cyano, oxo, or —N(R₄)—SO₂—R₅; R₄ and R₅ are each independently selected from H, C₁₋₆ alkyl or halo C₁₋₆ alkyl.

Further more, Y is

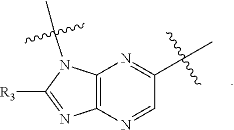

Further more, R₁ is selected from H or C₁₋₄ alkyl, preferably, R₁ is H, methyl, ethyl, propyl, isopropyl, n-butyl, sec-butyl, tert-butyl; and/or, R₂ is selected from H or C₁₋₃ alkyl; preferably, R₂ is H, methyl, ethyl, propyl or isopropyl; and/or, Q is absent or C₁₋₃ alkylene; preferably, Q is selected from methylene or ethylene; and/or, X is selected from H, C₁₋₃ alkyl or phenyl, alternatively, phenyl can be substituted with halogen, halo C₁₋₃ alkyl, C₁₋₃ alkyl, C₁₋₃ alkoxy or C₁₋₃ alkyl-SO₂—.

Further more, the compound is selected from one or more of the compound of formula II and the pharmaceutically acceptable salts, prodrugs, solvates and hydrates thereof:

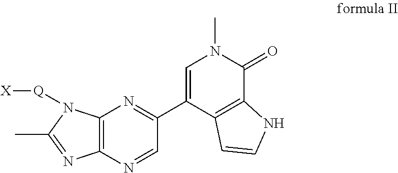

in the formula II, Q is C₁₋₆ alkylene; X is phenyl, the phenyl is optionally substituted with halogen, halo C₁₋₃ alkyl, C₁₋₃ alkyl, C₁₋₃ alkoxy or C₁₋₃ alkyl-SO₂—.

Further more, the compound is selected from one or more of:
6-methyl-4-(2-methyl-1-(4-(trifluoromethyl)benzyl)-1H-imidazo[4,5-b]pyrazin-6-yl)-1H-pyrrolo[2,3-c]pyridin-7(6H)-one;
4-(1-(4-chlorobenzyl)-2-methyl-1H-imidazo[4,5-b]pyrazin-6-yl)-6-methyl-1H-pyrrolo[2,3-c]pyridin-7(6H)-one;
4-(1-(4-methoxybenzyl)-2-methyl-1H-imidazo[4,5-b]pyrazin-6-yl)-6-methyl-1H-pyrrol o[2,3-c]pyridin-7(6H)-one;
4-(1-(4-methoxybenzyl)-2-methyl-1H-imidazo[4,5-b]pyrazin-6-yl)-6-methyl-1H-pyrrol o[2,3-c]pyridin-7(6H)-one;
4-(1-benzyl-2-methyl-1H-imidazo[4,5-b]pyrazin-6-yl)-6-methyl-1H-pyrrolo[2,3-c]pyri din-7(6H)-one;
4-(1-(3-trifluoromethylbenzyl)-2-methyl-1H-imidazo[4,5-b]pyrazin-6-yl)-6-methyl-1H-pyrrolo[2,3-c]pyridin-7(6H)-one;
4-(1-(2-fluoro-5-trifluoromethylbenzyl)-2-methyl-1H-imidazo[4,5-b]pyrazin-6-yl)-6-m ethyl-1H-pyrrolo[2,3-c]pyridin-7(6H)-one;
4-(1-(3-fluoro-5-trifluoromethylbenzyl)-2-methyl-1H-imidazo[4,5-b]pyrazin-6-yl)-6-m ethyl-1H-pyrrolo[2,3-c]pyridin-7(6H)-one;
4-(1-(2-fluoro-4-chlorobenzyl)-2-methyl-1H-imidazo[4,5-b]pyrazin-6-yl)-6-methyl-1H-pyrrolo[2,3-c]pyridin-7(6H)-one;
4-(1-(3-trifluoromethyl-4-chlorobenzyl)-2-methyl-1H-imidazo[4,5-b]pyrazin-6-yl)-6-m ethyl-1H-pyrrolo[2,3-c]pyridin-7(6H)-one;
4-(1-(3-fluoro-4-trifluoromethylbenzyl)-2-methyl-1H-imidazo[4,5-b]pyrazin-6-yl)-6-m ethyl-1H-pyrrolo[2,3-c]pyridin-7(6H)-one;
4-(1-(3-chloro-4-trifluoromethylbenzyl)-2-methyl-1H-imidazo[4,5-b]pyrazin-6-yl)-6-m ethyl-1H-pyrrolo[2,3-c]pyridin-7(6H)-one;
4-(1-(3-chlorobenzyl)-2-methyl-1H-imidazo[4,5-b]pyrazin-6-yl)-6-methyl-1H-pyrrolo[2,3-c]pyridin-7(6H)-one;
4-(1-(2,4-difluorobenzyl)-2-methyl-1H-imidazo[4,5-b]pyrazin-6-yl)-6-methyl-1H-pyrr olo[2,3-c]pyridin-7(6H)-one;
4-(1-(4-bromobenzyl)-2-methyl-1H-imidazo[4,5-b]pyrazin-6-yl)-6-methyl-1H-pyrrolo[2,3-c]pyridin-7(6H)-one;
6-methyl-4-(2-methyl-1-(4-(methylsulfonyl)benzyl)-1H-imidazo[4,5-b]pyrazin-6-yl)-1H-pyrrolo[2,3-c]pyridin-7(6H)-one;

preferably, the compound is 6-methyl-4-(2-methyl-1-(4-(trifluoromethyl)benzyl)-1H-imidazo[4,5-b]pyrazin-6-yl)-1H-py rrolo[2,3-c]pyridin-7(6H)-one.

Further more, the medicament also comprising excipients.

Further more, the medicament is administered intravenously, intramuscularly, parenterally, nasally, orally or rectally.

According to another aspect of the present invention, there is also provided a method of treating myelofibrosis and the symptoms/signs associated with myelofibrosis, wherein the method comprising: providing a medicament comprising the compound, wherein the compound is selected from one or more of the compound of formula I and the pharmaceutically acceptable salts, prodrugs, solvates and hydrates thereof; and administering therapeutically effective amount of the medicament to subject having myelofibrosis;

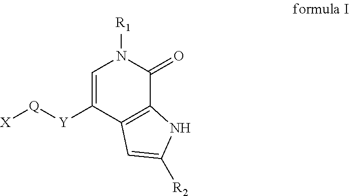

in the formula I,
R₁ and R₂ are each independently selected from H, C₁₋₆ alkyl, C₁₋₆ alkoxy, C₆₋₁₀ aryl or C₅₋₁₀ heteroaryl, wherein the C₁₋₆ alkyl, C₁₋₆ alkoxy, C₆₋₁₀ aryl or C₅₋₁₀ heteroaryl is optionally substituted with C₁₋₆ alkyl group, —NH₂ group, —OH group, C₆₋₁₀ aryl group or C₅₋₁₀ heteroaryl group; and the C₅₋₁₀ heteroaryl and the C₅₋₁₀ heteroaryl group has 1, 2, or 3 heteroatoms independently selected from nitrogen, oxygen or sulfur;
Q is absent or selected from C₁₋₆ alkylene, —SO₂— or —NH—, wherein the C₁₋₆ alkylene or —NH— is optionally substituted with halogen, C₁₋₆ alkyl or C₁₋₆ alkoxy;
X is selected from H, C₁₋₆ alkyl, C₆₋₁₀ aryl or C₅₋₁₀ heteroaryl, wherein the C₁₋₆ alkyl, C₆₋₁₀ aryl or C₅₋₁₀ heteroaryl is optionally substituted by halogen, halo C₁₋₆ alkyl, C₁₋₆ alkyl, C₁₋₆ alkoxy, C₁₋₆ alkylthio, C₁₋₆ alkoxycarbonyl or C₁₋₆ alkyl-SO₂—;
Y is

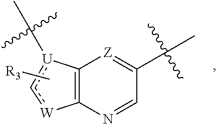

wherein,

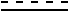

represents a single bond or a double bond, the U, W or Z is independently selected from C or N; R₃ is absent or selected from H, halogen, hydroxyl, amino, C₁₋₆ alkyl, C₁₋₆ alkoxy, cyano, oxo, or —N(R₄)—SO₂—R₅; R₄ and R₅ are each independently selected from H, C₁₋₆ alkyl or halo C₁₋₆ alkyl.

Further more, Y is

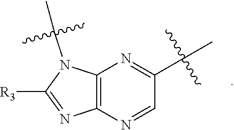

Further more, R₁ is selected from H or C₁₋₄ alkyl, preferably, R₁ is H, methyl, ethyl, propyl, isopropyl, n-butyl, sec-butyl, tert-butyl; and/or, R₂ is selected from H or C₁₋₃ alkyl; preferably, R₂ is H, methyl, ethyl, propyl or isopropyl; and/or, Q is absent or C₁₋₃ alkylene; preferably, Q is selected from methylene or ethylene; and/or, X is selected from H, C₁₋₃ alkyl or phenyl, alternatively, phenyl can be substituted with halogen, halo C₁₋₃ alkyl, C₁₋₃ alkyl, C₁₋₃ alkoxy or C₁₋₃ alkyl-SO₂—.

Further more, the compound is selected from one or more of the compound of formula II and the pharmaceutically acceptable salts, prodrugs, solvates and hydrates thereof:

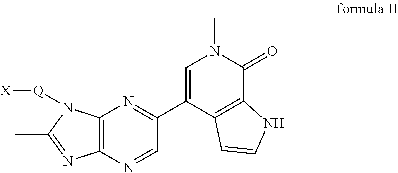

in the formula II, Q is C₁₋₆ alkylene; X is phenyl, the phenyl is optionally substituted with halogen, halo C₁₋₃ alkyl, C₁₋₃ alkyl, C₁₋₃ alkoxy or C₁₋₃ alkyl-SO₂—.

Further more, the compound is selected from one or more of:
6-methyl-4-(2-methyl-1-(4-(trifluoromethyl)benzyl)-1H-imidazo[4,5-b]pyrazin-6-yl)-1H-pyrrolo[2,3-c]pyridin-7(6H)-one;
4-(1-(4-chlorobenzyl)-2-methyl-1H-imidazo[4,5-b]pyrazin-6-yl)-6-methyl-1H-pyrrolo[2,3-c]pyridin-7(6H)-one;
4-(1-(4-methoxybenzyl)-2-methyl-1H-imidazo[4,5-b]pyrazin-6-yl)-6-methyl-1H-pyrrol o[2,3-c]pyridin-7(6H)-one;
4-(1-(4-methoxybenzyl)-2-methyl-1H-imidazo[4,5-b]pyrazin-6-yl)-6-methyl-1H-pyrrol o[2,3-c]pyridin-7(6H)-one;
4-(1-benzyl-2-methyl-1H-imidazo[4,5-b]pyrazin-6-yl)-6-methyl-1H-pyrrolo[2,3-c]pyri din-7(6H)-one;
4-(1-(3-trifluoromethylbenzyl)-2-methyl-1H-imidazo[4,5-b]pyrazin-6-yl)-6-methyl-1H-pyrrolo[2,3-c]pyridin-7(6H)-one;
4-(1-(2-fluoro-5-trifluoromethylbenzyl)-2-methyl-1H-imidazo[4,5-b]pyrazin-6-yl)-6-m ethyl-1H-pyrrolo[2,3-c]pyridin-7(6H)-one;
4-(1-(3-fluoro-5-trifluoromethylbenzyl)-2-methyl-1H-imidazo[4,5-b]pyrazin-6-yl)-6-m ethyl-1H-pyrrolo[2,3-c]pyridin-7(6H)-one;
4-(1-(2-fluoro-4-chlorobenzyl)-2-methyl-1H-imidazo[4,5-b]pyrazin-6-yl)-6-methyl-1H-pyrrolo[2,3-c]pyridin-7(6H)-one;
4-(1-(3-trifluoromethyl-4-chlorobenzyl)-2-methyl-1H-imidazo[4,5-b]pyrazin-6-yl)-6-m ethyl-1H-pyrrolo[2,3-c]pyridin-7(6H)-one;
4-(1-(3-fluoro-4-trifluoromethylbenzyl)-2-methyl-1H-imidazo[4,5-b]pyrazin-6-yl)-6-m ethyl-1H-pyrrolo[2,3-c]pyridin-7(6H)-one;
4-(1-(3-chloro-4-trifluoromethylbenzyl)-2-methyl-1H-imidazo[4,5-b]pyrazin-6-yl)-6-m ethyl-1H-pyrrolo[2,3-c]pyridin-7(6H)-one;
4-(1-(3-chlorobenzyl)-2-methyl-1H-imidazo[4,5-b]pyrazin-6-yl)-6-methyl-1H-pyrrolo[2,3-c]pyridin-7(6H)-one;
4-(1-(2,4-difluorobenzyl)-2-methyl-1H-imidazo[4,5-b]pyrazin-6-yl)-6-methyl-1H-pyrr olo[2,3-c]pyridin-7(6H)-one;
4-(1-(4-bromobenzyl)-2-methyl-1H-imidazo[4,5-b]pyrazin-6-yl)-6-methyl-1H-pyrrolo[2,3-c]pyridin-7(6H)-one;
6-methyl-4-(2-methyl-1-(4-(methylsulfonyl)benzyl)-1H-imidazo[4,5-b]pyrazin-6-yl)-1H-pyrrolo[2,3-c]pyridin-7(6H)-one;
   preferably, the compound is 6-methyl-4-(2-methyl-1-(4-(trifluoromethyl)benzyl)-1H-imidazo[4,5-b]pyrazin-6-y)-1H-py rrolo[2,3-c]pyridin-7(6H)-one.

Further more, the myelofibrosis is selected from the group consisting of primary myelofibrosis, Post-Polycythemia Vera Myelofibrosis or Post-Essential Thrombocythemia Myelofibrosis.

Further more, the medicament also comprising excipients.

Further more, the medicament is administered intravenously, intramuscularly, parenterally, nasally, orally or rectally.

Further more, the compound is administered in a dosage of 1-500 mg/day.

Further more, the compound is administered orally in a dosage of about 3 mg/day, or 5 mg/day, or 10 mg/day, or 20 mg/day, or 25 mg/day, or 30 mg/day, or 35 mg/day, or 40 mg/day, or 45 mg/day, or 50 mg/day, or 55 mg/day, or 60 mg/day, or 70 mg/day, or 80 mg/day, or 90 mg/day, or 100 mg/day, or 150 mg/day, or 200 mg/day.

The present invention provides a use of the compound in the preparation of a medicament for treating myelofibrosis and the symptoms/signs associated with myelofibrosis; the compound is selected from one or more of the compound of formula I and the pharmaceutically acceptable salts, prodrugs, solvates and hydrates thereof:

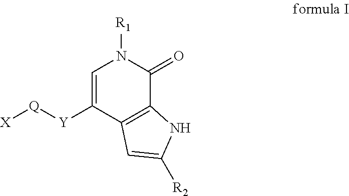

the compound has a significant inhibitory effect on the proliferation of human myeloproliferative tumor cells HEL and SET-2 cells with JAK2V617F mutation phenotype, and also has a good inhibitory effect on the transcriptional activity of NF-κB and the secretion of the inflammatory factor IL-6, and can be useful in the treatment of myelofibrotic disease and the symptoms/signs associated with myelofibrosis.

### DESCRIPTION OF THE DRAWINGS

The drawings that constitute a part of the present invention are used to provide a further understanding, the illustrative examples and there descriptions are used to explain the present invention and do not constitute an improper limitation of the present invention.

In the drawings:

FIG. 1 shows the proliferation inhibition curve of compound 1 against HEL cells at different concentrations in Example 2:

FIG. 2 shows the proliferation inhibition curve of compound 1 against SET-2 cells at different concentrations in Example 2;

FIG. 3 shows the inhibition curve of compound 1 on NF-κB transcriptional activity at different concentrations in Example 3;

FIG. 4 shows the inhibitory curve of compound 1 on IL-6 in THP-1 cells at different concentrations in Example 4.

### EXAMPLES

It should be noted that, as long as there is no conflict, the examples and features in the examples of the present invention can be combined with each other. The present invention will be described in detail below with reference to the drawings and examples.

### Definitions

In the present invention, unless otherwise stated, the term “halogen” refers to fluorine, chlorine, bromine or iodine. Preferably, halogen refers to fluorine, chlorine and bromine.

In the present invention, unless otherwise stated, the term “alkyl” includes a straight, branched or cyclic saturated monovalent hydrocarbon. For example, alkyl includes methyl, ethyl, propyl, isopropyl, cyclopropyl, n-butyl, isobutyl, sec-butyl, tert-butyl, cyclobutyl, n-pentyl, 3-(2-methyl)butyl, 2-pentyl, 2-methylbutyl, neopentyl, cyclopentyl, n-hexyl, 2-hexyl, 2-methylpentyl and cyclohexyl. Similarly, “C₁₋₆” in C₁₋₆ alkyl refers to a group containing 1, 2, 3, 4, 5 or 6 carbon atoms arranged in a linear or branched chain.

In the present invention, unless otherwise stated, the term “heteroaryl” refers to a substituted or unsubstituted stable monocyclic or bicyclic group containing at least one aromatic ring of 5 to 10 ring atoms, the aromatic ring containing one, two or three ring heteroatoms selected from N, O and S, the remaining ring atoms are C atoms. Examples of such heteroaryl groups include, but are not limited to, pyridyl, pyrimidinyl, pyrrolyl, imidazolyl, thiazolyl, thienyl, and benzimidazole.

In the present invention, the term “prodrugs” refers to functional derivatives of the compounds that are readily converted in vivo into the required compound. Thus, in the methods for the treatment of the present invention, the term “administering” shall encompass the treatment of the various disorders described with the compound specifically disclosed or with a compound which may not be specifically disclosed, but which converts to the specified compound in vivo after administration to the subject, for the treatment of myelofibrosis and the symptoms/signs associated with myelofibrosis.

When the compound of Formula I and pharmaceutically acceptable salts thereof exist in the form of solvates or polymorphic forms, the “compound” of the present invention includes any possible solvates and polymorphic forms. A type of a solvent that forms the solvate is not particularly limited so long as the solvent is pharmacologically acceptable. For example, water, ethanol, propanol, acetone and similar solvents can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, formic, hydrochloric, maleic, phosphoric, sulfuric and tartaric acids, particularly preferred are formic and hydrochloric acid. Since the compounds of Formula I are intended for pharmaceutical use they are preferably provided in substantially pure form, for example at least 60% pure, more suitably at least 75% pure, especially at least 98% pure (% are on a weight for weight basis).

In order to treat myelofibrosis and its associated symptoms/signs with medicament, the present invention provides a use of the compound in the preparation of a medicament for treating myelofibrosis and the symptoms/signs associated with myelofibrosis, and provides a method of treating myelofibrosis and the symptoms/signs associated with myelofibrosis. The above method comprising: providing a medicament comprising the compound, and administering therapeutically effective amount of the medicament to subject having myelofibrosis. The above compound is selected from one or more of the compound of formula I and the pharmaceutically acceptable salts, prodrugs, solvates and hydrates thereof:

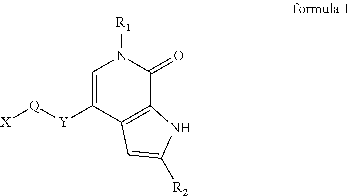

in the formula I,
R₁ and R₂ are each independently selected from H, C₁₋₆ alkyl, C₁₋₆ alkoxy, C₆₋₁₀ aryl or C₅₋₁₀ heteroaryl, wherein the C₁₋₆ alkyl, C₁₋₆ alkoxy, C₆₋₁₀ aryl or C₅₋₁₀ heteroaryl is optionally substituted with C₁₋₆ alkyl group, —NH₂ group, —OH group, C₆₋₁₀ aryl group or C₅₋₁₀ heteroaryl group; and the C₅₋₁₀ heteroaryl and the C₅₋₁₀ heteroaryl group have 1, 2, or 3 heteroatoms independently selected from nitrogen, oxygen or sulfur;
Q is absent or selected from C₁₋₆ alkylene, —SO₂— or —NH—, wherein the C₁₋₆ alkylene or —NH— is optionally substituted with halogen, C₁₋₆ alkyl or C₁₋₆ alkoxy;
X is selected from H, C₁₋₆ alkyl, C₆₋₁₀ aryl or C₅₋₁₀ heteroaryl, wherein the C₁₋₆ alkyl, C₆₋₁₀ aryl or C₅₋₁₀ heteroaryl is optionally substituted by halogen, halo C₁₋₆ alkyl, C₁₋₆ alkyl, C₁₋₆ alkoxy, C₁₋₆ alkylthio, C₁₋₆ alkoxycarbonyl or C₁₋₆ alkyl-SO₂—;
Y is

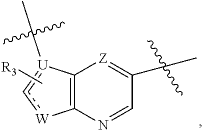

wherein,

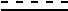

represents a single bond or a double bond, the U, W or Z is independently selected from C or N; R₃ is absent or selected from H, halogen, hydroxyl, amino, C₁₋₆ alkyl, C₁₋₆ alkoxy, cyano, oxo, or —N(R₄)—SO₂—R₅; R₄ and R₅ are each independently selected from H, C₁₋₆alkyl or halo C₁₋₆ alkyl.

The inventors of the present invention unexpectedly discovered that the compound of Formula I has a significant inhibitory effect on the proliferation of human myeloproliferative tumor cells HEL and SET-2 cells with JAK2V617F mutation phenotype, and also has a good inhibitory effect on the transcriptional activity of NF-κB and the secretion of the inflammatory factor IL-6, its associated symptoms/signs, and can be useful in the treatment of myelofibrotic disease and its associated symptoms/signs.

In addition, it should be noted that, the Y group of the aboved compound of formula I is required to be

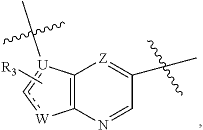

compared to other 5- and/or 6-membered merged rings, such as

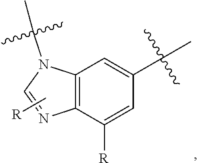

the compound of formula I of the present invention has significant differences and obvious effectiveness in the treatment of myelofibrosis. More preferably, Y is

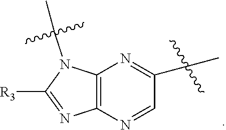

In order to further improving the effectiveness of treatment of myelofibrosis and the symptoms/signs associated with myelofibrosis, in the preferred embodiment, R₁ is selected from H or C₁₋₆ alkyl, preferably, R₁ is H, methyl, ethyl, propyl, isopropyl, n-butyl, sec-butyl, tert-butyl; more preferably. R₁ is methyl.

In a preferred embodiment, R₂ is selected from H or C₁₋₃ alkyl; preferably, R₂ is H, methyl, ethyl, propyl or isopropyl; more preferably, R₂ is H.

In a preferred embodiment, Q is absent or C₁₋₃ alkylene; more preferably, Q is selected from methylene or ethylene; more preferably, Q is methylene.

In a preferred embodiment, X is selected from H, C₁₋₃ alkyl or phenyl, alternatively, phenyl can be substituted with halogen, halo C₁₋₃ alkyl, C₁₋₃ alkyl, C₁₋₃ alkoxy or C₁₋₃ alkyl-SO₂—.

In a preferred embodiment, the compound is selected from one or more of the compound of formula II and the pharmaceutically acceptable salts, prodrugs, solvates and hydrates thereof:

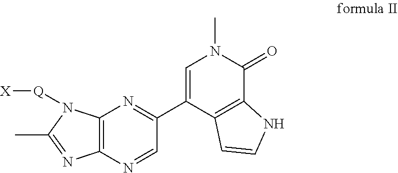

in the formula II, Q is C₁₋₆ alkylene; X is phenyl, the phenyl is optionally substituted with halogen, halo C₁₋₃ alkyl, C₁₋₃ alkyl, C₁₋₃ alkoxy or C₁₋₃ alkyl-SO₂—.

Illustratively, the above compounds are selected from one or more of:
6-methyl-4-(2-methyl-1-(4-(trifluoromethyl)benzyl)-1H-imidazo[4,5-b]pyrazin-6-yl)-1H-pyrrolo[2,3-c]pyridin-7(6H)-one;
4-(1-(4-chlorobenzyl)-2-methyl-1H-imidazo[4,5-b]pyrazin-6-yl)-6-methyl-1H-pyrrolo[2,3-c]pyridin-7(6H)-one;
4-(1-(4-methoxybenzyl)-2-methyl-1H-imidazo[4,5-b]pyrazin-6-yl)-6-methyl-1H-pyrrol o[2,3-c]pyridin-7(6H)-one;
4-(1-(4-methoxybenzyl)-2-methyl-1H-imidazo[4,5-b]pyrazin-6-yl)-6-methyl-1H-pyrrol o[2,3-c]pyridin-7(6H)-one;
4-(1-benzyl-2-methyl-1H-imidazo[4,5-b]pyrazin-6-yl)-6-methyl-1H-pyrrolo[2,3-c]pyri din-7(6H)-one;
4-(1-(3-trifluoromethylbenzyl)-2-methyl-1H-imidazo[4,5-b]pyrazin-6-yl)-6-methyl-1H-pyrrolo[2,3-c]pyridin-7(6H)-one;
4-(1-(2-fluoro-5-trifluoromethylbenzyl)-2-methyl-1H-imidazo[4,5-b]pyrazin-6-yl)-6-m ethyl-1H-pyrrolo[2,3-c]pyridin-7(6H)-one;
4-(1-(3-fluoro-5-trifluoromethylbenzyl)-2-methyl-1H-imidazo[4,5-b]pyrazin-6-yl)-6-m ethyl-1H-pyrrolo[2,3-c]pyridin-7(6H)-one;
4-(1-(2-fluoro-4-chlorobenzyl)-2-methyl-1H-imidazo[4,5-b]pyrazin-6-yl)-6-methyl-1H-pyrrolo[2,3-c]pyridin-7(6H)-one:
4-(1-(3-trifluoromethyl-4-chlorobenzyl)-2-methyl-1H-imidazo[4,5-b]pyrazin-6-yl)-6-m ethyl-1H-pyrrolo[2,3-c]pyridin-7(6H)-one;
4-(1-(3-fluoro-4-trifluoromethylbenzyl)-2-methyl-1H-imidazo[4,5-b]pyrazin-6-yl)-6-m ethyl-1H-pyrrolo[2,3-c]pyridin-7(6H)-one;
4-(1-(3-chloro-4-trifluoromethylbenzyl)-2-methyl-1H-imidazo[4,5-b]pyrazin-6-yl)-6-m ethyl-1H-pyrrolo[2,3-c]pyridin-7(6H)-one;
4-(1-(3-chlorobenzyl)-2-methyl-1H-imidazo[4,5-b]pyrazin-6-yl)-6-methyl-1H-pyrrolo[2,3-c]pyridin-7(6H)-one;
4-(1-(2,4-difluorobenzyl)-2-methyl-1H-imidazo[4,5-b]pyrazin-6-yl)-6-methyl-1H-pyrr olo[2,3-c]pyridin-7(6H)-one;
4-(1-(4-bromobenzyl)-2-methyl-1H-imidazo[4,5-b]pyrazin-6-yl)-6-methyl-1H-pyrrolo[2,3-c]pyridin-7(6H)-one;
6-methyl-4-(2-methyl-1-(4-(methylsulfonyl)benzyl)-1H-imidazo[4,5-b]pyrazin-6-yl)-1H-pyrrolo[2,3-c]pyridin-7(6H)-one;
   preferably, the compound is 6-methyl-4-(2-methyl-1-(4-(trifluoromethyl)benzyl)-1H-imidazo[4,5-b]pyrazin-6-yl)-1H-py rrolo[2,3-c]pyridin-7(6H)-one.

In one embodiment of the invention, the symptoms/signs associated with myelofibrosis is one or more of the following: anemia, splenomegaly, hepatomegaly, fatigue, early satiety, abdominal discomfort, inactivity, inability to concentrate, night sweats, itching, diffuse bone pain, fever, and weight loss.

In practical applications, the medicament also comprises excipients. Specific excipients include, but not limited to, pharmaceutically acceptable carriers, excipients or adjuvants, etc. Moreover, the medicament can be prepared into different dosage forms to adapt to diverse administration route, the specific suitable administration routes include intravenous, intramuscular, parenterally, nasally, orally or rectally. The dosage forms of the medicament include tablets, capsules, pills, powders, suppositories, solutions, suspensions, aerosols, emulsions, ointments, lotions, dusting powders or other similar dosage forms.

The pharmaceutical carrier employed can be, for example, a solid, liquid, or gas. Examples of solid carriers include such as lactose, terra alba, sucrose, talc, gelatin, agar, pectin, acacia, magnesium stearate, and stearic acid. Examples of liquid carriers include such as sugar syrup, peanut oil, olive oil, and water. Examples of gaseous carriers include such as carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

The above tablet may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient. For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 1 mg, 1.5 mg, 2 mg, 2.5 mg, 3 mg, 5 mg, 15 mg, 20 mg, 25 mg, 50 mg, 100 mg, 200 mg, 300 mg, 400 mg, 500 mg, 600 mg, 800 mg or 1000 mg.

The above mentioned medicament suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

The above mentioned medicament suitable for injectable use includes sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

The above mentioned medicament can be in a form suitable for rectal administration with solid as a carrier. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

The term “effective amount” is therapeutically effective amount. The “therapeutically effective amount” is an amount sufficient to provide a desired therapeutic result over the necessary dosage and specified duration. The therapeutically effective amount of the medicament can vary depending on various factors, for example, the individual's condition, age, gender, and weight, as well as the ability of drugs to elicit the desired response in the individual's body. The therapeutically effective amount is also the amount in which the therapeutically beneficial effects outweigh any toxic or detrimental effects of the subject. In one embodiment, in the method of treating myelofibrosis and the symptoms/signs associated with myelofibrosis provided by the present invention, the above compound is administered in dosage of 1-500 mg/day, preferably the dosage of the compound is 3˜200 mg/day, preferably the dosage of the compound is 5˜150 mg/day. Exemplarily, the above compound is administered in dosage of about 3 mg/day, or 5 mg/day, or 10 mg/day, or 20 mg/day, or 25 mg/day, or 30 mg/day, or 35 mg/day, or 40 mg/day, or 45 mg/day, or 50 mg/day, or 55 mg/day, or 60 mg/day, or 70 mg/day, or 80 mg/day, or 90 mg/day, or 100 mg/day, or 150 mg/day, or 200 mg/day for oral administration.

The present invention will be described in detail below in conjunction with examples, which are merely illustrative and not limited to the scope of application of the present invention.

### ABBREVIATIONS

ALT: Alanine Aminotransferase;
ANC: Absolute Neutrophil Count;
APTT: Activated Partial Thromboplastin Time;
AST: Aspartate Transaminase;
BID: Twice A Day;
CI: Clinical Improvement;
CT: Computed Tomography;
CR: Complete Response;
Ccr: Creatinine Clearance;
cm: Centimeter,
DIEA: N,N-diisopropylethylamine;
DIPSS: Dynamic International Prognostic Scoring System;
DLTs/DLT: Dose-Limiting Toxicities;
DMSO: Dimethyl Sulfoxide;
DCM: Dichloromethane;
EA: Ethyl Acetate;
ECOG: Eastern Cooperative Oncology Group;
ELN: European Leukemia Network;
HGB: Hemoglobin;
INR: International Normalized Ratio;
IWG-MRT: International Working Group-Myeloproliferative Neoplasms Research and Treatment;
LCMS/LC-MS: Liquid Chromatography Coupled With Mass Spectrometry;
MF: Myelofibrosis;
MPN: Myeloproliferative Neoplasm;
MPN-SAF: MPN Symptom Assessment Form;
MPN-SAF TSS: MPN Symptom Assessment Form-Total Symptom Score;
MTD: Maximum Tolerated Dose;
MRI: Nuclear Magnetic Resonance;
mg: Milligram;
mL: Milliliter,
mM: Millimole Per Liter;
nM: Nanomoles Per Liter;
μM: Micromoles Per Liter;
NCI CTCAE: National Cancer Institute Common Terminology Criteria for Adverse Events:
Pd(dppf)Cl₂.DCM: [1,1′-bis(diphenylphosphine)ferrocene]Palladium Dichloride Dichloromethane Complex;
RP2D: Phase II recommended Dose;
PE: Petroleum Ether;
PMF: Primary Myelofibrosis;
post-PV MF: Post-Polycythemia Vera Myelofibrosis;
post-ET MF: Post-Essential Thrombocythemia Myelofibrosis;
PLT: Platelet;
PR: Partial Response;
PD: Progressive Disease;
Scr: Serum Creatinine;
SD: Stable Disease;
TBIL: Serum Total Bilirubin;
ULN: Upper Limit Of Normal;
WHO: World Health Organization.

### Example 1

Preparation of 6-methyl-4-(2-methyl-1-(4-(trifluoromethyl)benzyl)-1H-imidazo[4,5-b]pyrazin-6-yl)-1H-py rrolo[2,3-c]pyridin-7(6H)-one

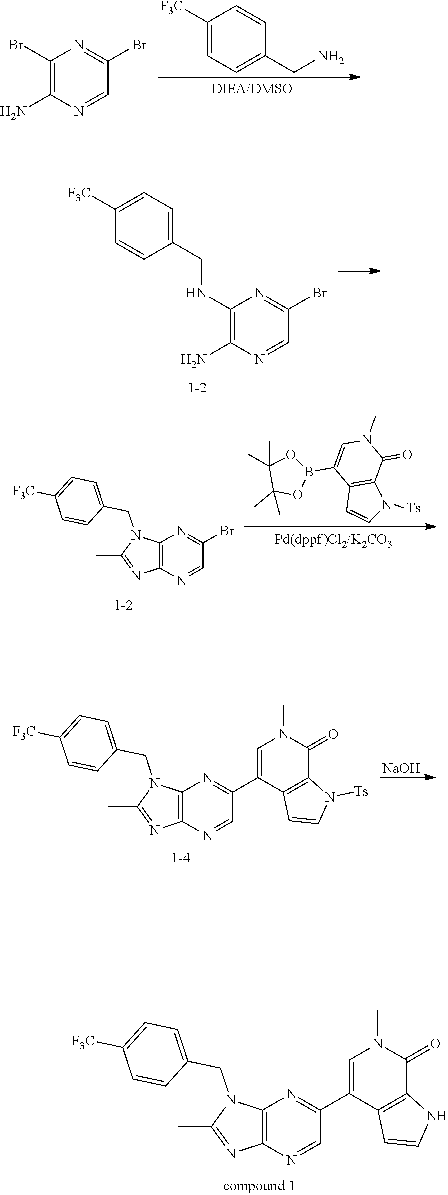

### Step 1. Synthesis of Compound 1-I

2-amino-3,5-dibromopyrazine (10.00 g), 4-trifluoromethylbenzylamine (20.98 g) and DIEA (25.54 g) was dissolved in DMSO (60 mL), heated and stirred at 120° C. for 4 h, LCMS confirmed the end of the reaction. Cooled, cold water (250 mL) was added, extracted 3 times with EA, combined organic phases, washed with saturated brine 3 times, dried with anhydrous sodium sulfate, concentrated, and purified the crude product by column chromatography (the mobile phase is PE:EA=100:15-100:30) to obtain compound 1-1(15.01 g).

LCMS: [M+1]⁺=347.0

### Step 2. Synthesis of Compound 1-2

Compound 1-1 (15.43 g), triethyl orthoacetate (35.96 g) and glacial acetic acid (200 mL) were mixed and reacted at 100° C. overnight. Cooled, concentrated, diluted the crude product with EA, wash 3 times with saturated Na₂CO₃ solution, 3 times with saturated brine, dried over anhydrous sodium sulfate, and concentrated. The crude product was purified by column chromatography (the mobile phase is PE:EA=100:20-100:50) to obtain compound 1-2 (13.27 g).

LCMS: [M+1]⁺=371.0

### Step 3, Synthesis of Compound 1-4

Compound 1-2 (1.10 g), 2M−1(1.27 g), Pd(dppf)Cl₂.DCM(0.25 g) were dissolved in dioxane (20 mL), K₂CO₃ (0.61 g) and water (4 mL) were added, protected by nitrogen, heated and stirred at 100° C. overnight. Cooled, the reaction solution was poured into a mixed solvent of EA (50 mL) and saturated Na₂CO₃ (50 mL), separated the liquid, aqueous phase was extracted 3 times with EA, organic phases were combined, washed with saturated brine 3 times, dried over anhydrous sodium sulfate, and concentrated, the crude product was purified by column chromatography (the mobile phase is DCM:MeOH=100:2) to obtain compound 1-4 (1 g).

LCMS: [M+1]⁺=593.1

### Step 4. Synthesis of Compound 1

Compound 1-4 (20.00 g) was dissolved in EtOH (80 mL) and DCM (250 mL), NaOH (2.70 g) was added, stirred at 60° C. overnight, concentrated. The crude product was purified by column chromatography (the mobile phase is DCM:MeOH=100:2) to obtain a brownish-yellow crude product, the brownish-yellow crude product was slurried with DCM (40 mL), the solid was collected by filtration, and dried under reduced pressure to give compound 1 as a off-white solid (6.53 g).

LCMS: [M+1]⁺=439.1.

¹H NMR (400 MHz, DMSO) δ 12.13 (s, 1H), 8.92 (s, 1H), 7.99 (d, J=35.1 Hz, 1H), 7.76 (t, J=17.0 Hz, 2H), 7.49 (d, J=8.0 Hz, 2H), 7.28 (s, 1H), 6.75 (s, 1H), 5.68 (s, 2H), 3.63 (s, 3H), 2.63 (s, 3H).

Using a method basically similar to that of Example 1, the corresponding trifluoromethylbenzylamine derivative is used to replace

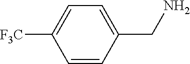 [SMILES: NCc1ccc(C(F)(F)F)cc1]

(trifluoromethylbenzylamine) to prepare the example in Table 1 below. The corresponding trifluoromethylbenzylamine derivative, such as

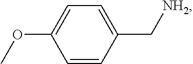

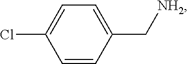

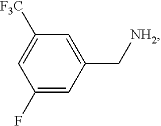 [SMILES: NCc1cc(F)cc(C(F)(F)F)c1]

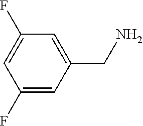

or

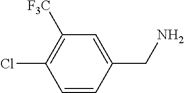 [SMILES: NCc1ccc(Cl)c(C(F)(F)F)c1]

etc, can all be purchased through commercially available channels.

TABLE 1

| Ex. | | | Physical data |
|---|---|---|---|
| No. | Structure | Chemical Name | (MS) (M + H)⁺ |
| 2 | 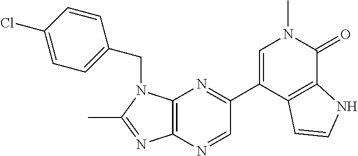 [SMILES: Cc1nc2ncc(-c3cn(C)c(=O)c4[nH]ccc34)nc2n1Cc1ccc(Cl)cc1] | 4-(1-(4-chlorobenzyl)-2-methyl- 1H-imidazo[4,5-b]pyrazin- 6-yl)-6-methyl-1H-pyrrolo[2,3-c] pyridin-7(6H)-one | 405.8 |
| 3 | 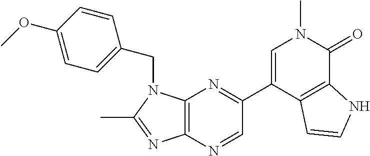 [SMILES: COc1ccc(Cn2c(C)nc3ncc(-c4cn(C)c(=O)c5[nH]ccc45)nc32)cc1] | 4-(1-(4-methoxybenzyl)-2-methyl- 1H-imidazo[4,5-b]pyrazin-6-yl)- 6-methyl-1H-pyrrolo[2,3-c] pyridin-7(6H)-one | 401.2 |
| 4 | 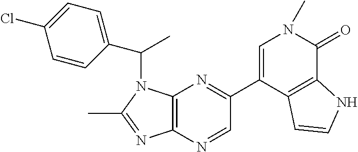 [SMILES: Cc1nc2ncc(-c3cn(C)c(=O)c4[nH]ccc34)nc2n1C(C)c1ccc(Cl)cc1] | 4-(1-(1-(4-chlorophenyl)ethyl)- 2-methyl-1H-imidazo[4,5-b] pyrazin-6-yl)-6-methyl-1,6- dihydro-7H-pyrrolo[2,3-c] pyridin-7-one | 419.1 |
| 5 | 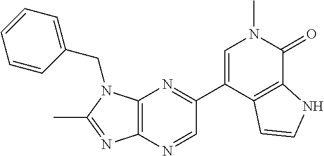 [SMILES: Cc1nc2ncc(-c3cn(C)c(=O)c4[nH]ccc34)nc2n1Cc1ccccc1] | 4-(1-benzyl-2-methyl-1H- imidazo[4,5-b]pyrazin-6-yl)- 6-methyl-1H-pyrrolo[2,3-c] pyridin-7(6H)-one | 371.1 |
| 6 | 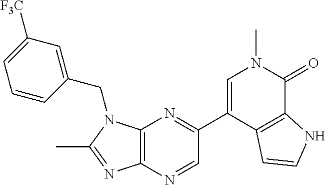 [SMILES: Cc1nc2ncc(-c3cn(C)c(=O)c4[nH]ccc34)nc2n1Cc1cccc(C(F)(F)F)c1] | 4-(1-(3-trifluoromethylbenzyl)- 2-methyl-1H-imidazo[4,5-b] pyrazin-6-yl)-6-methyl-1H- pyrrolo[2,3-c]pyridin-7(6H)-one | 439.1 |
| 7 | 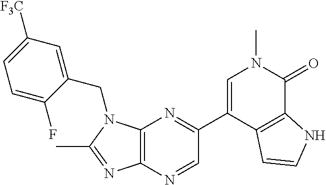 [SMILES: Cc1nc2ncc(-c3cn(C)c(=O)c4[nH]ccc34)nc2n1Cc1cc(C(F)(F)F)ccc1F] | 4-(1-(2-fluoro-5-trifluoromethyl- benzyl)-2-methyl-1H-imidazo [4,5-b]pyrazin-6-yl)-6-methyl- 1H-pyrrolo[2,3-c]pyridin-7(6H)- one | 457.1 |
| 8 | 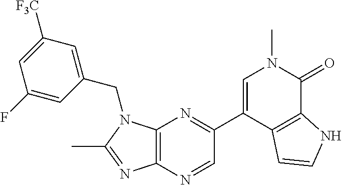 [SMILES: Cc1nc2ncc(-c3cn(C)c(=O)c4[nH]ccc34)nc2n1Cc1cc(F)cc(C(F)(F)F)c1] | 4-(1-(3-fluoro-5-trifluoromethyl- benzyl)-2-methyl-1H-imidazo [4,5-b]pyrazin-6-yl)-6-methyl- 1H-pyrrolo[2,3-c]pyridin-7(6H)- one | 457.1 |
| 9 | 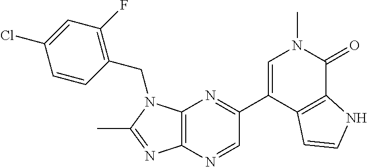 [SMILES: Cc1nc2ncc(-c3cn(C)c(=O)c4[nH]ccc34)nc2n1Cc1ccc(Cl)cc1F] | 4-(1-(2-fluoro-4-chlorobenzyl)- 2-methyl-1H-imidazo[4,5-b] pyrazin-6-yl)-6-methyl-1H- pyrrolo[2,3-c]pyridin-7(6H)- one | 423.1 |
| 10 | 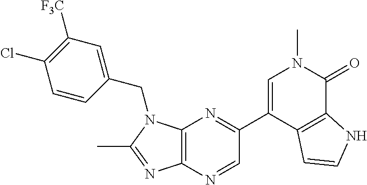 [SMILES: Cc1nc2ncc(-c3cn(C)c(=O)c4[nH]ccc34)nc2n1Cc1ccc(Cl)c(C(F)(F)F)c1] | 4-(1-(3-trifluoromethyl-4-chloro- benzyl)-2-methyl-1H-imidazo [4,5-b]pyrazin-6-yl)-6-methyl- 1H-pyrrolo[2,3-c]pyridin-7(6H)- one | 473.1 |
| 11 | 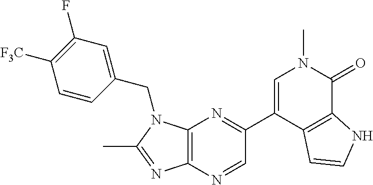 [SMILES: Cc1nc2ncc(-c3cn(C)c(=O)c4[nH]ccc34)nc2n1Cc1ccc(C(F)(F)F)c(F)c1] | 4-(1-(3-fluoro-4-trifluoromethyl- benzyl)-2-methyl-1H-imidazo [4,5-b]pyrazin-6-yl)-6-methyl- 1H-pyrrolo[2,3-c]pyridin-7(6H)- one | 457.1 |
| 12 | 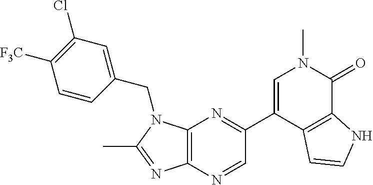 [SMILES: Cc1nc2ncc(-c3cn(C)c(=O)c4[nH]ccc34)nc2n1Cc1ccc(C(F)(F)F)c(Cl)c1] | 4-(1-(3-chloro-4-trifluoromethyl- benzyl)-2-methyl-1H-imidazo [4,5-b]pyrazin-6-yl)-6-methyl- 1H-pyrrolo[2,3-c]pyridin-7(6H)- one | 473.1 |
| 13 | 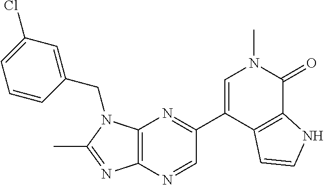 [SMILES: Cc1nc2ncc(-c3cn(C)c(=O)c4[nH]ccc34)nc2n1Cc1cccc(Cl)c1] | 4-(1-(3-chlorobenzyl)-2-methyl- 1H-imidazo[4,5-b]pyrazin- 6-yl)-6-methyl-1H-pyrrolo [2,3-c]pyridin-7(6H)-one | 405.1 |
| 14 | 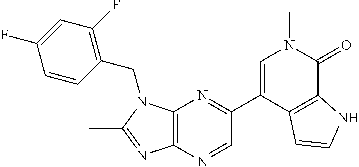 [SMILES: Cc1nc2ncc(-c3cn(C)c(=O)c4[nH]ccc34)nc2n1Cc1ccc(F)cc1F] | -(1-(2,4-difluorobenzyl)-2-methyl- 1H-imidazo[4,5-b]pyrazin- 6-yl)-6-methyl-1H-pyrrolo [2,3-c]pyridin-7(6H)-one | 407.1 |
| 15 | 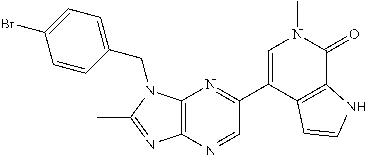 [SMILES: Cc1nc2ncc(-c3cn(C)c(=O)c4[nH]ccc34)nc2n1Cc1ccc(Br)cc1] | 4-(1-(4-bromobenzyl)-2-methyl- 1H-imidazo[4,5-b]pyrazin- 6-yl)-6-methyl-1H-pyrrolo[2,3-c] pyridin-7(6H)-one | 449.06 |
| 16 | 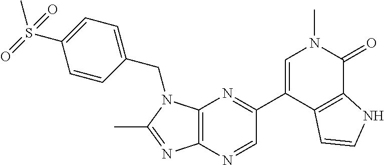 [SMILES: Cc1nc2ncc(-c3cn(C)c(=O)c4[nH]ccc34)nc2n1Cc1ccc(S(C)(=O)=O)cc1] | 6-methyl-4-(2-methyl-1-(4- (methylsulfonyl)benzyl)-1H- imidazo[4,5-b]pyrazin-6-yl)- 1H-pyrrolo[2,3-c]pyridin-7(6H)- one | 449.1 |

### Example 2

The effects of compound 1 on proliferation of human myeloproliferative tumor cells HEL and SET-2 cells with JAK2V617F activation mutation were tested using CTG assay in this example to evaluate half inhibitory rate (IC₅₀) of compound 1 on HEL and SET-2 cell proliferation.

### Method

### 1. Cell Culture:

a) HEL cells were cultured in RPMI 1640 medium containing 1% PS and 10% FBS. The cells were incubated in a humidified incubator with 5% carbon dioxide (CO₂) at 37° C.
b) SET-2 cells were cultured in RPMI 1640 medium containing 1% PS and 20% heat-inactive FBS. The cells were incubated in a humidified incubator with 5% carbon dioxide (CO₂) at 37° C.

### 2. Cell Plating:

a) Cells were harvested upon reaching a certain density.
b) Cells were resuspended in corresponding medium with a certain density.
c) 30 μL of cell suspension was added into each well of a 384-well plate. For HEL, the density was 1800 cells/well, and for SET-2, 2000 cells/well.
d) Cells were incubated overnight in a humidified incubator with 5% CO₂ at 37° C.

### 3. Compound Preparation

The compounds were diluted with DMSO 6.67 fold from 20 mM to 3 mM, then 3-fold serial dilutions were conducted to make 10 concentrations for each compound. Staurosporine was diluted with DMSO 6.67 fold from 20 mM to 3 mM, followed by 3-fold serial dilutions to make 10 concentrations. The vehicle control consisted of 100% DMSO, and the positive control was 3 mM Staurosporine.

### 4. Compound Treatment

a) 24 hours post cell seeding, 100 nL of diluted compound from step 3 was added to each well of the 384-well plates.
b) The final concentrations of 2 compounds and Staurosporine were: 10000, 3333, 1111, 370.4, 123.5, 41.2, 13.7, 4.6, 1.5, 0.5 nM.
c) The positive control had a final concentration of 10 μM Staurosporine, and the vehicle control had a final concentration of 0.33% DMSO.
d) Cells were incubated for 72 h in a 37° C., 5% CO₂ incubator.

### 5. CTG Detection

a) 30 μL CTG reagent (CelltiterGlo kit) was added to each well, and the plate was subjected to orbital shaking for 2 minutes, then incubated at room temperature in the dark for 30 minutes.
b) Luminescence values were obtained using Envision.

### 6. Data Analysis

IC₅₀ (the half maximal inhibitory concentration) was calculated with GraphPad Prism 8 software and following nonlinear fitting formula:

$$Y = Bottom + ( Top - Bottom ) / ( 1 + 10 \hat{} ( ( Log {IC}_{50} - X ) * HillSlope ) )$$

$$X : Log value of Compound concentration , \\ Y : percentage inhibition rate ( \% inhibition )$$

$$\% Inhibition = ( {LUM}_{High} - {LUM}_{cmpd} ) / ( {LUM}_{High} - {LUM}_{Low} ) * 100$$

LUM_{High} is the average value of 0.33% DMSO wells, LUM_{empd} is the value of each well of the compound, LUM_{Low} is the average value of 10 μM Staurosporine wells.

### Results:

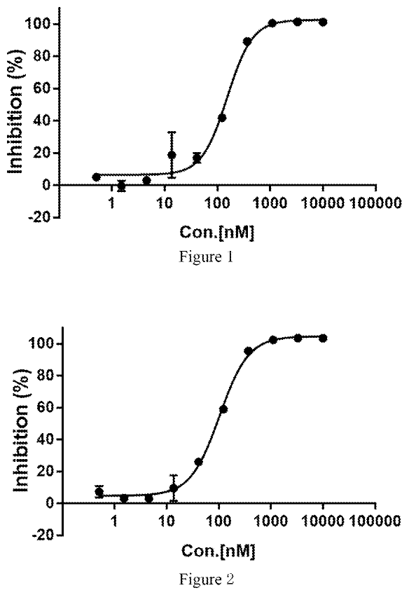

The proliferation inhibition curve of compound 1 against HEL cells at different concentrations is shown in FIG. 1, and the proliferation inhibition curve against SET-2 cells is shown in FIG. 2.

The IC₅₀ values of compound 1 against HEL cells and SET-2 cells are 154.1 nM and 103.6 nM, respectively. These results indicate that compound 1 exhibits a good inhibitory effect on the proliferation of both HEL and SET-2 cells.

TABLE 2
IC₅₀ (nM) for cell proliferation inhibition

| Cell lines | Compound | IC₅₀(nM) | Hillslope | Bottom | Top |
|---|---|---|---|---|---|
| HEL | Compound 1 | 154.1 | 1.8 | 6.4 | 102.5 |
| SET-2 | Compound 1 | 103.6 | 1.6 | 4.7 | 104.6 |

### Example 3

In present example, reporter gene assay was used to determine the inhibitory effect of compound 1 on NF-κB transcriptional activity in HEK293 cells, to evaluate the effect of the compound on NF-κB transcriptional activity.

### Method:

### 1. Compound Preparation

a) All compounds were 3-fold serial diluted from 10 mM for 10 doses in DMSO.
b) The positive compound CDDO-Me was 3-fold serial diluted from 10 mM for 10 doses in DMSO.
c) 1000×positive control (100r DMSO) and 1000×negative control (100% DMSO) were prepared.
d) Compounds plate was sealed and shaked for 5 minutes.

### 2. Cell Suspension Preparation

a) All cells were cultured following ATCC recommendations. HEK293T-NFκB cells were assayed during exponential growth phase.
b) The culture medium was removed from the flask, and then the cells were rinsed with PBS.
c) The cells were digested with trypsin. Digestion was terminated with complete culture medium, and the cells were collected and counted.
d) A total of 6*10⁶ HEK293T cells were seeded into a 100 mm cell culture dish.
e) The cells were then incubated overnight in a 5% CO₂ incubator.

### 3. Compound Treatment

a) 25 nL diluted compounds from Step 1 were transferred to the cell culture plate with Echo 655.
b) Cells (prepared in step2) were seeded into the 384-well assay plate at a density of 17,000 cells/well. The seeding medium used was phenol red-free DMEM containing 5% charcoal-stripped FBS and 8 ng/ml TNFα.
c) The cells were incubated for 16-20 h in a 5% CO₂ incubator at 37° C.

### 4. Compound Detection

a) The Britelite plus reagent were allowed to reach room temperature.
b) The 384-well assay plates (prepared at Step 3) were allowed to reach room temperature.
c) 25 μL Britelite plus reagent was added to each well of 384-well assay plate.
d) Luminescence values were recorded with Envision.

### 5. Data Analysis

### 5.1 the Percent Inhibition was Calculated as Follows:

$$\% Inhibition = 100 - ⌊ \frac{R L {U}_{Compound} - {\overline{R L U}}_{Positive}}{{\overline{R L U}}_{Vehicle} - {\overline{R L U}}_{Positive}} ⌋ * 100$$

RLU: relative luminescence units
R̅L̅U̅_{Positive}: The average luminescence value of the blank controls.
R̅L̅U̅_{vehicle}: The average luminescence value of the negative controls.
5.2 IC₅₀ calculating and compound dose-response curves fitting:

The following nonlinear fitting formula was used to calculate the IC₅₀ of the compound with Graphpad8.0.

$$Y = Bottom + ( Top - Bottom ) / ( 1 + 10 \hat{} ( ( Log {IC}_{50} - X ) * HillSlope ) )$$

$$X : Log value of compound concentration ; \\ Y : Compound percentage inhibition$$

### Results:

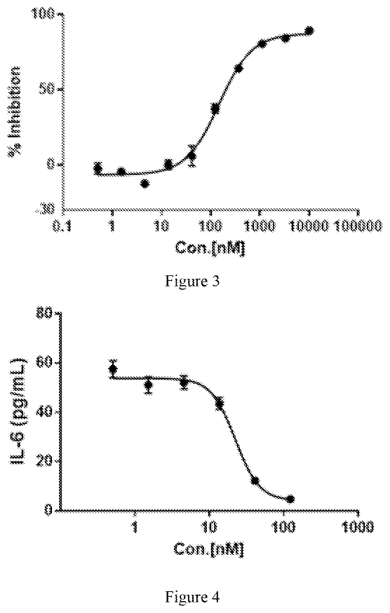

The inhibition curve of compound 1 on NF-κB transcriptional activity at different concentrations was shown in FIG. 3.

Under the conditions of this experiment, compound 1 significantly inhibited the transcriptional activity of NF-κB in HEK293 cells stably transfected with the NF-κB reporter gene.

TABLE 3
IC₅₀ (nM) for the inhibition of NF-κB transcriptional activity

| Compound | IC₅₀(nM) | Hillslope | Bottom | Top |
|---|---|---|---|---|
| Compound 1 | 147.6 | 1.3 | −6.5 | 87.4 |

### Example 4

The Human IL-6 Valukine ELISA kit (purchased from R&D) was used to measure the IL-6 content in the cell supernatants by ELISA method in this example. The experimental procedures shown below were conducted to determine the effect of compound 1 on IL-6 secretion in THP-1 cell supernatants following LPS stimulation.

### Method:

### 1. Cell Culture:

THP-1 cells were cultured in RPMI 1640 medium containing 1% PS and 10% FBS and 2-Mercaptoethanol (IX). The cells were incubated in a humidified incubator with 5% carbon dioxide (CO₂) at 37° C.

### 2. Cell Plating:

a) The cells were harvested when the confluence reached 80%/90%.
b) Subsequently, the cells were resuspended to a certain density with RPMI Medium 1640 containing 1% PS and 10% heat-inactive FBS.
c) The cell suspension was added to a 96-well plate, 100 μL per well, and the cell density was 150,000/well.
d) The cells were incubated in a humidified incubator with 5% CO₂ at 37° C.

### 3. Compound Preparation

a) Compound 1 was diluted 2 fold with DMSO from 20 mM to 10 mM, followed by further 3-fold serial dilutions to generate 9 concentrations.
b) Vehicle control was 100% DMSO.
c) A working stock solution was made by adding 5 μL of the serial-diluted compound to 45 μL culture medium.

### 4. Compound Treatment

a) 96 μL medium and 2 μL compound working stock solution from Step 3 were added to each well.
b) After 30 minutes incubation, 2 μL LPS was adde to each well, resulting in a final concentration of 4 μg/mL.
c) The final testing concentrations of compound 1 were: 10000, 3333, 1111, 370.4, 123.5, 41.2, 13.7, 4.6, 1.5, 0 nM.
d) The final vehicle control comprised 0.1% DMSO, the blank control was cells without LPS treatment.
e) The cells were incubated in a 5% CO₂ incubator at 37° C.

### 5. IL-6 ELISA Detection

a) 96-well plate was centrifuged for 5 minutes at 1500 rpm, and pipetted 150 μL/well cellular supernatant from each well.
b) Before proceeding, all reagents were left to equilibrate to room temperature and mixed thoroughly to avoid foaming.
c) Determine the number of strips required based on the number of experimental wells (blanks and standards).
d) Add samples: 100 μL diluted standards and test samples were added to each well, sealed and incubated for 2 hours at room temperature.
e) Washing: The liquid in the well was removed, 300 μL 1×Wash Buffer was added to each well; discarded the liquid in the well after staying for 1 minute. Repeated 3 times, dried it on filter paper each time.
f) Add detection antibody: 200 μL Human IL-6 Conjugate was added to each well, then sealed and incubated for 2 hours at room temperature.
g) Washing: Repeated Step e).
h) Reaction: 200 μL Substrate Solution was added to each well, incubated for 20 minutes at room temperature, in dark conditions.
i) Termination reaction: 50 μL Stop solution was added to each well quickly to terminate the reaction.
j) Plate reading: Within 30 minutes after termination, read the value using the measurement wavelength 450 nm.

### 6. CTG Detection

a) 50 μL medium was added to each well.
b) 100 μL CTG reagent (CelltiterGlo kit) was added to each well, and the plate was placed on a orbital shaker for 2 minutes, then placed at room temperature in dark conditions for 30 minutes, and then transferred to CTG plate.
c) The luminescence values were recorded with Envision.

### 7. Data Analysis

IC₅₀ (The half maximal inhibitory concentration) was calculated using GraphPad Prism 8 software with the following nonlinear fitting formula:

$$Y = Bottom + ( Top - Bottom ) / ( 1 + 10 \hat{} ( ( Log {IC}_{50} - X ) * HillSlope ) )$$

a) CTG detection: X: Log value of Compound concentration, Y: cell viability inhibition rate (Inhibition %)

$$Inhibition \% = ( 1 - Compd / Vehicle control ) * 100$$

b) IL-6 detection: X: Log value of Compound concentration, Y: IL-6 concentration (pg/mL)
IL-6 concentration was calculated based on the linear equation of standard dilution concentration and wavelength detection.

$$Plate 1 : y = 0.0117 x + 0.0421 {R}^{2} = 0.9998$$

$$Plate 2 : y = 0.0111 x + 0.0445 {R}^{2} = 0.9996$$

Note: When the cell viability inhibition rate is greater than 30%, IL-6 at this concentration dose not participate in the calculation of IC₅₀.

### Results:

The inhibitory curve of compound 1 on IL-6 in THP-1 cells at different concentrations was shown in FIG. 4.

As compound concentration increased, the concentration of IL-6 in the supernatant of THP-1 cells decreased, indicating that compound 1 significantly inhibited the IL-6 secretion in THP1 monocytes.

TABLE 4
IC₅₀ (nM) for inhibition of IL-6 secretion in THP-1 cells

| Compound | IC₅₀(nM) | Hillslope | Bottom | Top |
|---|---|---|---|---|
| Compound 1 | 22.4 | −2.7 | 4.2 | 53.9 |

The above data demonstrated that the compound of the present invention displayed a significant inhibitory effect on the proliferation of human myeloproliferative tumor cell lines HEL and SET-2 harboring the JAK2V617F mutantion, which is directly associated with myelofibrosis. Furthermore, the compound exhibited a substantial inhibitory effect on the transcriptional activity of NF-κB and the secretion of the inflammatory factor IL-6.

### Example 5

A Phase I study to evaluate safety, tolerability, pharmacokinetics and efficacy of compound 1 and determine MTD and RP2D

Target: Subjects with primary myelofibrosis, post-polycythemia vera myelofibrosis, post-essential thrombocythemia myelofibrosis.

### Inclusion Criteria:

1. Male or female adults, aged ≥18 years;
2. Subjects have diagnosis of primary myelofibrosis (PMF) according to WHO criteria or diagnosis of post-polycythemia vera myelofibrosis (post-PV MF) or post-essential thrombocythemia myelofibrosis (post-ET MF) according to IWG-MRT criteria;
3. Dose escalation phase: Myelofibrosis (MF) patients have “intermediate-1 risk” and above according to Dynamic International Prognostic Scoring System (DIPSS) (with one or more adverse prognostic factors) and have received at least one therapy, patients cannot benefit from existing therapy or the patient has been assessed by the Investigator as not being a suitable candidate or otherwise ineligible for the standard of care therapy.
4. Dose expansion phase: Myelofibrosis (MF) patients have “intermediate-1 risk” and above according to Dynamic International Prognostic Scoring System (DIPSS) (with one or more adverse prognostic factors);
5. The expanded enrollment study requires patients with symptomatic splenomegaly: Palpate the edge of the spleen to reach or exceed at least 5 cm below the ribs (the distance from the rib edge to the farthest point of the spleen);
6. Expected survival ≥24 weeks months;
7. Eastern Cooperative Oncology Group (ECOG) performance status 0-2;
8. Summation of all the individual scores ≥1, evaluated by the MPN Symptoms Assessment Form(MPN-SAF);
9. Bone marrow and peripheral blood blast count ≤20%;
10. Patients who have not had splenic radiotherapy within 6 months before screening;
11. Adequate organ function defined as:
a) Blood routine: Absolute neutrophil count (ANC)≥1.0×10⁹/L, platelets (PLT)≥100×10⁹/L, hemoglobin (HGB) ≥7.5 g/dL (75 g/L) (No red blood cell transfusions within 14 days before the screening test);
b) Coagulation function: Subjects who are not receiving anticoagulation therapy, international normalized ratio (INR) and activated partial thromboplastin time (APTT)≤1.5 times the upper limit of normal values(ULN);
c) Liver: Serum total bilirubin (TBIL)≤2.0×ULN, if the subjects have Gilbert syndrome, TBIL is required to be ≤3.0×ULN; aspartate aminotransferase (AST) and alanine aminotransferase (ALT) ≤2.5×ULN;
d) Kidneys: Serum creatinine (Scr) ≤1.5×ULN or creatinine clearance (Ccr) ≥50 mL/min (calculated by Cockcroft-Gault formula);
12. The toxic reactions of previous anti-tumor treatments have returned to the severity before treatment or NCI CTCAE version 4.03≤grade 1 (the same is true for alopecia or other toxicities that are considered to have no safety risk to the patient in neonates);
13. Women of child-bearing potential must be non-lactating period, and the result of a serum pregnancy test performed within 7 days before starting treatment must be negative; all subjects must use medically approved contraceptive measures during the entire treatment and within 3 months after the last dose of study drug;
14. Subjects must comply with the study protocol and follow-up procedures;
15. Subjects must have signed written, informed consent.

### Dose Escalation Study Design:

This study used a “3+3” design and designed 3 dose levels initially, that is, after determining the initial dose of 60 mg, the test doses were increased by 33% and 25%, which were 60 mg/d, 80 mg/d and 100 mg/d respectively. “3+3” design escalation rule: \textcircled{1} If no DLTs are observed of 3 DLT-evaluable patients, the trial proceeds to next higher dose cohort; \textcircled{2}If one subject develops a DLT of 3 DLT-evaluable patients, an additional three subjects are enrolled into that same dose cohort, once 31 DLTs are observed further (total number of DLT patients≥2), the maximum tolerated dose (MTD) will be declared as the previous dose level; \textcircled{3} If 2 or more patients in a cohort experience DLTs, then stop enrollment at the specific dose and higher dose levels, the MTD will be declared as the previous dose level.

Twice daily (BID, Q12H was used in this study, two doses per day every 12 hours), then the dose level and dosing regimens (including, but not limited to, starting dose, intended escalating maximum dose, intermediate dose, dosing frequency and intervals) can be adjusted by the sponsor and researcher based on the result of pharmacokinetics, safety, tolerability and efficacy. 21 days each treatment cycle.

### Efficacy Evaluation Method:

a) Routine blood test and peripheral blood smear
b) Bone marrow biopsy or bone marrow cytology smear
c) MRI
d) Palpation of spleen
e) Spleen ultrasound
f) Evaluation of myelofibrosis degree
g) Evaluation of bone marrow hyperplasia grade

### Efficacy Evaluation Criteria:

The 2013 ELN and IWG-MRT consensus standards were adopted.

TABLE 5
Efficacy evaluation criteria

| Response categories | Required criteria |
|---|---|
| Complete | All the following categories must be qualified: |
| Remission | (1) Bone marrow: Age-adjusted normocellularity; <5% blasts; ≤grade 1 |
| (CR) | MF(European classification); |
| | (2) Peripheral blood: HGB ≥100 g/L, ANC ≥1 × 10⁹/L; PLT ≥100 × 10⁹/L, |
| | none of the above indicators have reached upper normal limit; <2% |
| | immature myeloid cells; |
| | (3) Clinical: Resolution of disease symptoms; spleen and liver not |
| | palpable; no evidence of extramedullary hematopoiesis. |
| Partial | Belong to one of the following categories: |
| Remission | (1) Peripheral blood: HGB ≥100 g/L, ANC ≥1 × 10⁹/L, PLT ≥100 × 10⁹/L, |
| (PR) | none of the above indicators have reached upper normal limit; <2% |
| | immature myeloid cells; Clinical: Resolution of disease symptoms; |
| | spleen and liver not palpable; no evidence of extramedullary |
| | hematopoiesis. |
| | (2) Bone marrow: Age-adjusted normocellularity; <5% blasts; ≤grade |
| | 1 MF and peripheral blood: HGB (85~<100) g/L, ANC ≥1 × 10⁹/L, PLT |
| | (50~<100) × 10⁹/L, none of the above indicators have reached upper |
| | normal limit; <2% immature myeloid cells; Clinical: Resolution of |
| | disease symptoms; spleen and liver not palpable; complete resolution of |
| | physical signs, including hepatosplenomegaly; no evidence of |
| | extramedullary hematopoiesis. |
| Clinical | The achievement of anemia, splenomegaly or symptoms response |
| improvement(CI) | without progressive disease increase in severity of anemia, |
| | thrombocytopenia, or neutropenia. |
| | Anemia response: Transfusion-independent patients: a ≥20 g/L |
| | increase in hemoglobin level; Transfusion-dependent patients: becoming |
| | transfusion-independent (absence of red blood cells transfusions during |
| | the treatment phase and HGB ≥85 g/L). |
| | Spleen response: (1) A baseline splenomegaly at 5-10 cm below the left |
| | costal margin becomes not palpable; (2) A baseline splenomegaly that is |
| | palpable at >10 cm, below the left costal margin, decreases by ≥50%; |
| | (3) A baseline splenomegaly that is palpable at <5 cm, below the |
| | left costal margin, is not eligible for spleen response; (4) A spleen |
| | response requires confirmation by MRI or computed tomography |
| | showing ≥35% spleen volume reduction. |
| | Symptoms response: A ≥50% reduction in the MPN-SAF TSS. |
| Progressive | Belong to one of the following categories: |
| disease(PD)(PD) | (1) Appearance of a new splenomegaly that is palpable at least 5 cm |
| | below the left costal margin; |
| | (2) A ≥100% increase in palpable distance, below left costal margin, |
| | for baseline splenomegaly of 5-10 cm; |
| | (3) A >50% increase in palpable distance, below left costal margin, for |
| | baseline splenomegaly of >10 cm; |
| | (4) Leukemic transformation confirmed by a bone marrow blast count |
| | of ≥20%; |
| | (5) A peripheral blood blast content of ≥20% associated with an |
| | absolute blast count of ≥1 × 10(9)/L that lasts for at least 2 weeks. |
| Stable | Belonging to none of the above listed response categories. |
| disease (SD) | Belong to one of the following categories: |
| Relapse | (1) No longer meeting criteria for at least CI after achieving CR, PR, or |
| | CI; |
| | (2) Loss of anemia response persisting for at least 1 month; |
| | (3) Loss of spleen response persisting for at least 1 month. |
| Cytogenetic | At least 10 metaphases must be analyzed for cytogenetic response |
| remission | evaluation and requires confirmation by repeat testing within 6 months |
| | window. |
| | (1) CR: Eradication of a preexisting cytogenetic abnormalities; |
| | (2) PR: ≥50% reduction in preexisting abnormal metaphases (partial |
| | response applies only to patients with at least ten abnormal metaphases |
| | at baseline). |
| Molecular | Molecular response evaluation must be analyzed in peripheral blood |
| remission | granulocytes and requires confirmation by repeat testing within 6 months |
| | window. |
| | (1) CR: Eradication of a preexisting molecular biology; |
| | (2) PR: ≥50% decrease in allele burden(partial response applies only to |
| | patients with at least 20% mutant allele burden at baseline). |
| Cytogenetic/ | Re-emergence of a pre-existing cytogenetic or molecular abnormality |
| molecular relapse | that is confirmed by repeat testing |

The above descriptions are only preferred embodiments of the present invention, and are not intended to limit the present invention. For those skilled in the art, the present invention may have various modifications and changes. Any modifications, equivalent substitutions, improvements, etc. made within the spirit and principles of the present invention shall be included in the protection scope of the present invention.

1-7. (canceled)

8. A method of treating myelofibrosis and the symptoms/signs associated with myelofibrosis, wherein the method comprising:
providing a medicament comprising the compound, wherein the compound is selected from one or more of the compound of formula I and the pharmaceutically acceptable salts, prodrugs, solvates and hydrates thereof, and administering therapeutically effective amount of the medicament to subject having myelofibrosis; 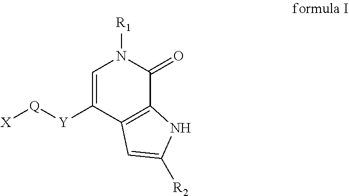
in the formula I,
R₁ and R₂ are each independently selected from H, C₁₋₆ alkyl, C₁₋₆ alkoxy, C₆₋₁₀ aryl or C₅₋₁₀ heteroaryl, wherein the C₁₋₆ alkyl, C₁₋₆ alkoxy, C₆₋₁₀ aryl or C₅₋₁₀ heteroaryl is optionally substituted with C₁₋₆ alkyl group, —NH₂ group, —OH group, C₆₋₁₀ aryl group or C₅₋₁₀ heteroaryl group; and the C₅₋₁₀ heteroaryl and the C₅₋₁₀ heteroaryl group has 1, 2, or 3 heteroatoms independently selected from nitrogen, oxygen or sulfur;
Q is absent or selected from C₁₋₆ alkylene, —SO₂— or —NH—, wherein the C₁₋₆ alkylene or —NH— is optionally substituted with halogen, C₁₋₆ alkyl or C₁₋₆ alkoxy;
X is selected from H, C₁₋₆ alkyl, C₆₋₁₀ aryl or C₅₋₁₀ heteroaryl, wherein the C₁₋₆ alkyl, C₆₋₁₀ aryl or C₅₋₁₀ heteroaryl is optionally substituted by halogen, halo C₁₋₆ alkyl, C₁₋₆ alkyl, C₁₋₆ alkoxy, C₁₋₆ alkylthio, C₁₋₆ alkoxycarbonyl or C₁₋₆ alkyl-SO₂—;
Y is 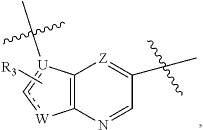
wherein, 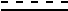
represents a single bond or a double bond, the U, W or Z is independently selected from C or N; R₃ is absent or selected from H, halogen, hydroxyl, amino, C₁₋₆ alkyl, C₁₋₆ alkoxy, cyano, oxo, or —N(R₄)—SO₂—R₅; R₄ and R₅ are each independently selected from H, C₁₋₆ alkyl or halo C₁₋₆ alkyl.

9. The method of claim 8, wherein, Y is

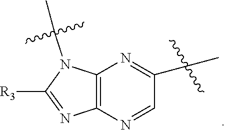

10. The method of claim 8, wherein,
R₁ is selected from H or C₁₋₄ alkyl; and/or
R₂ is selected from H or C₁₋₃ alkyl; and/or
Q is absent or C₁₋₃ alkylene; and/or
X is selected from H, C₁₋₃ alkyl or phenyl, alternatively, phenyl can be substituted with halogen, halo C₁₋₃ alkyl, C₁₋₃ alkyl, C₁₋₃ alkoxy or C₁₋₃ alkyl-SO₂—.

11. The method of claim 8, wherein, the compound is selected from one or more of the compound of formula II and the pharmaceutically acceptable salts, prodrugs, solvates and hydrates thereof: 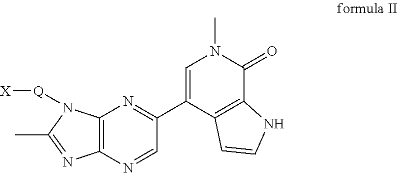
in the formula II,
Q is C₁₋₆ alkylene;
X is phenyl, the phenyl is optionally substituted with halogen, halo C₁₋₃ alkyl, C₁₋₃ alkyl, C₁₋₃ alkoxy or C₁₋₃ alkyl-SO₂—.

12. The method of claim 8, wherein, the compound is selected from one or more of:
6-methyl-4-(2-methyl-1-(4-(trifluoromethyl)benzyl)-1H-imidazo[4,5-b]pyrazin-6-yl)-1H-pyrrolo[2,3-c]pyridin-7(6H)-one;
4-(1-(4-chlorobenzyl)-2-methyl-1H-imidazo[4,5-b]pyrazin-6-yl)-6-methyl-1H-pyrrolo[2,3-c]pyridin-7(6H)-one;
4-(1-(4-methoxybenzyl)-2-methyl-1H-imidazo[4,5-b]pyrazin-6-yl)-6-methyl-1H-pyrrolo[2,3-c]pyridin-7(6H)-one;
4-(1-benzyl-2-methyl-1H-imidazo[4,5-b]pyrazin-6-yl)-6-methyl-1H-pyrrolo[2,3-c]pyridin-7(6H)-one;
4-(1-(3-trifluoromethylbenzyl)-2-methyl-1H-imidazo[4,5-b]pyrazin-6-yl)-6-methyl-1H-pyrrolo[2,3-c]pyridin-7(6H)-one;
4-(1-(2-fluoro-5-trifluoromethylbenzyl)-2-methyl-1H-imidazo[4,5-b]pyrazin-6-yl)-6-methyl-1H-pyrrolo[2,3-c]pyridin-7(6H)-one;
4-(1-(3-fluoro-5-trifluoromethylbenzyl)-2-methyl-1H-imidazo[4,5-b]pyrazin-6-yl)-6-methyl-1H-pyrrolo[2,3-c]pyridin-7(6H)-one;
4-(1-(2-fluoro-4-chlorobenzyl)-2-methyl-1H-imidazo[4,5-b]pyrazin-6-yl)-6-methyl-1H-pyrrolo[2,3-c]pyridin-7(6H)-one;
4-(1-(3-trifluoromethyl-4-chlorobenzyl)-2-methyl-1H-imidazo[4,5-b]pyrazin-6-yl)-6-methyl-1H-pyrrolo[2,3-c]pyridin-7(6H)-one;
4-(1-(3-fluoro-4-trifluoromethylbenzyl)-2-methyl-1H-imidazo[4,5-b]pyrazin-6-yl)-6-methyl-1H-pyrrolo[2,3-c]pyridin-7(6H)-one;
4-(1-(3-chloro-4-trifluoromethylbenzyl)-2-methyl-1H-imidazo[4,5-b]pyrazin-6-yl)-6-methyl-1H-pyrrolo[2,3-c]pyridin-7(6H)-one;
4-(1-(3-chlorobenzyl)-2-methyl-1H-imidazo[4,5-b]pyrazin-6-yl)-6-methyl-1H-pyrrolo[2,3-c]pyridin-7(6H)-one;
4-(1-(2,4-difluorobenzyl)-2-methyl-1H-imidazo[4,5-b]pyrazin-6-yl)-6-methyl-1H-pyrrolo[2,3-c]pyridin-7(6H)-one;
4-(1-(4-bromobenzyl)-2-methyl-1H-imidazo[4,5-b]pyrazin-6-yl)-6-methyl-1H-pyrrolo[2,3-c]pyridin-7(6H)-one; and
6-methyl-4-(2-methyl-1-(4-(methylsulfonyl)benzyl)-1H-imidazo[4,5-b]pyrazin-6-yl)-1H-pyrrolo[2,3-c]pyridin-7(6H)-one.

13. The method of claim 8, wherein, the medicament also comprises excipients.

14. The method of claim 8, wherein, the medicament is administered intravenously, intramuscularly, parenterally, nasally, orally or rectally.

15. The method of claim 8, wherein the compound is administered in a dosage of 1˜500 mg/day.

16. The method of claim 10, wherein R₁ is H, methyl, ethyl, propyl, isopropyl, n-butyl, sec-butyl or tert-butyl.

17. The method of claim 10, wherein R₂ is H, methyl, ethyl, propyl or isopropyl.

18. The method of claim 10, wherein Q is methylene or ethylene.

19. The method of claim 12, wherein the compound is 6-methyl-4-(2-methyl-1-(4-(trifluoromethyl)benzyl)-1H-imidazo[4,5-b]pyrazin-6-yl)-1H-pyrrolo[2,3-c]pyridin-7(6H)-one.

20. The method of claim 15, wherein the compound is administered orally in a dosage of 3 mg/day, or 5 mg/day, or 10 mg/day, or 20 mg/day, or 25 mg/day, or 30 mg/day, or 35 mg/day, or 40 mg/day, or 45 mg/day, or 50 mg/day, or 55 mg/day, or 60 mg/day, or 70 mg/day, or 80 mg/day, or 90 mg/day, or 100 mg/day, or 150 mg/day, or 200 mg/day.
